(19) United States
(12) Patent Application Publication
(10) Pub. No.: US 2016/0375264 A1
(43) Pub. Date: Dec. 29, 2016

(54) LIGHT WAVE TREATMENT INSTRUMENT AND METHODS OF USE

(71) Applicant: Edgar Dan Laperriere, Cookeville, TN (US)

(72) Inventor: Edgar Dan Laperriere, Cookeville, TN (US)

(21) Appl. No.: 15/176,436

(22) Filed: Jun. 8, 2016

Related U.S. Application Data
(60) Provisional application No. 62/183,931, filed on Jun. 24, 2015.

(51) Int. Cl. A61N 5/06; A61B 5/00; A61B 5/024

(52) U.S. Cl. CPC A61N 5/0603; A61N 5/0613; A61B 5/024; A61B 5/4836; A61N 2005/0606; A61N 2005/0629; A61N 2005/0651; A61N 2005/0654; A61N 2005/0659; A61N 2005/067

(57) ABSTRACT

A light wave treatment instrument is disclosed and generally comprises a control unit operably coupled to a power source; a light source operably coupled to the control unit operable to deliver pulses of optical radiation; and a heart rate monitor operably coupled to the control unit, wherein the light source delivers pulses of optical radiation to an area of desired treatment synchronized to the heart beat determined by the heart rate monitor.

### CROSS-REFERENCE TO RELATED APPLICATIONS

The present application claims priority to U.S. Provisional Application Ser. No. 62/183,931, filed Jun. 24, 2015, which is herein incorporated by reference in its entirety.

### BACKGROUND

The invention generally relates to light therapy.

Light therapy involves irradiating tissues with light. Light can stimulate a variety of biological activities in cells and tissues that are compromised in function. Light therapy treatment is typically administered by a physician or therapist who directs light from a hand-held light emitting device at an affected area. Light emitting devices can be difficult to position consistently over the affected area. Sometimes a tattoo is used to identify the affected area. However, even with a tattoo or other reference mark it is difficult to consistently deliver light therapy treatments to an affected area.

Light therapy typically involves repeated treatments over at least several days. Thus, patients undergoing light therapy may be required to make multiple visits to a practitioner's office or clinic in order to complete a therapy regimen. Such repeated visits may be time consuming and/or expensive.

Many modern people suffer from periodontal diseases. Generally, periodontal tissues include gum and alveolar bone, that is, periodontal tissues are the structure around a tooth. Its role is like a foundation of a building, and the tooth is like a main body of the building. The periodontal diseases can be compared to the foundation breaking down, although the main body of the building has no problem, the building may collapse at any moment because continuous loss of debris leads to stagger of the building.

Periodontal diseases can be substantially divided into two stages in accordance with the severity. In the initial stage, the periodontal disease is gingivitis, that is, inflammation is confined to the gum, which usually refers to tissues around the teeth turn very red or even swell. When the sufferer brushes his/her teeth, the swelling gum may be bloody. If the gingivitis is not treated, the gingivitis will be easily converted to periodontitis (i.e., periodontal disease) after a long time. By the medical research, it is known that, the periodontal diseases are mainly caused by plaques in tooth necks. When the plaques begin to release toxins, the supporting tissues around teeth will be violated by the toxins. These bacteria causing the periodontal disease multiply in large quantities, which will result in pain of the gum. When the periodontal disease become very serious, the gum will fester. If there is not any treatment, the periodontal disease will continuously worsen, and the bone of teeth will gradually lose. As a result, the teeth begin to shake, and then fall off one by one.

In traditional dental technology for treating periodontal diseases, substantially, the purpose is to reduce oral bacteria and decrease inflammation phenomenon of periodontal tissues (gum, periodontal ligament and so on). Generally, oral bacteria are scraped off, and antibiotics and anti-inflammatory drugs are used. A method for scraping off oral bacteria is to use a laser. Since the laser with a high energy is selected, teeth or periodontal tissues may be damaged when treating. After the traditional treatment, because of detumescence of the gum, the gum will atrophy, teeth root will be exposed, and the teeth are sensitive to cold and heat and so on.

The present invention attempts to solve these problems as well as others.

### SUMMARY OF THE INVENTION

Provided herein are systems, methods and apparatuses for a light wave treatment instrument. An light wave treatment instrument is disclosed and generally comprises a control unit operably coupled to a power source; a light source operably coupled to the control unit operable to deliver pulses of optical radiation; and a heart rate monitor operably coupled to the control unit, wherein the light source delivers pulses of optical radiation to an area of desired treatment synchronized to the heart beat determined by the heart rate monitor.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 is a schematic view of the light wave treatment instrument.

FIG. 2 is a side view of the optical couplers.

FIG. 3 is a perspective view of the light array.

FIG. 4 is a perspective view of the light array when illuminated with optical radiation.

FIG. 5A is a periodontal table measuring pocket depth of teeth for disease on Apr. 29, 2015.

FIG. 5B is a periodontal table measuring pocket depth of teeth for disease on May 15, 2015.

FIG. 5C is a periodontal table measuring pocket depth of teeth for disease on Jun. 20, 2015.

FIG. 6A is periodontal table showing the summary data comparison of the periodontal examination on Apr. 29, 2015.

FIG. 6B is periodontal table showing the summary data comparison of the periodontal examination on May 15, 2015.

FIG. 6C is periodontal table showing the summary data comparison of the periodontal examination on Jun. 20, 2015.

FIGS. 7A-7F are photos were taken associated with the Periodontal Examination.

FIGS. 8A-8H are photos of the positive results and reduction of the periodontal disease after phototherapy by the light wave treatment instrument.

FIG. 9 is a diagram of a dentition.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

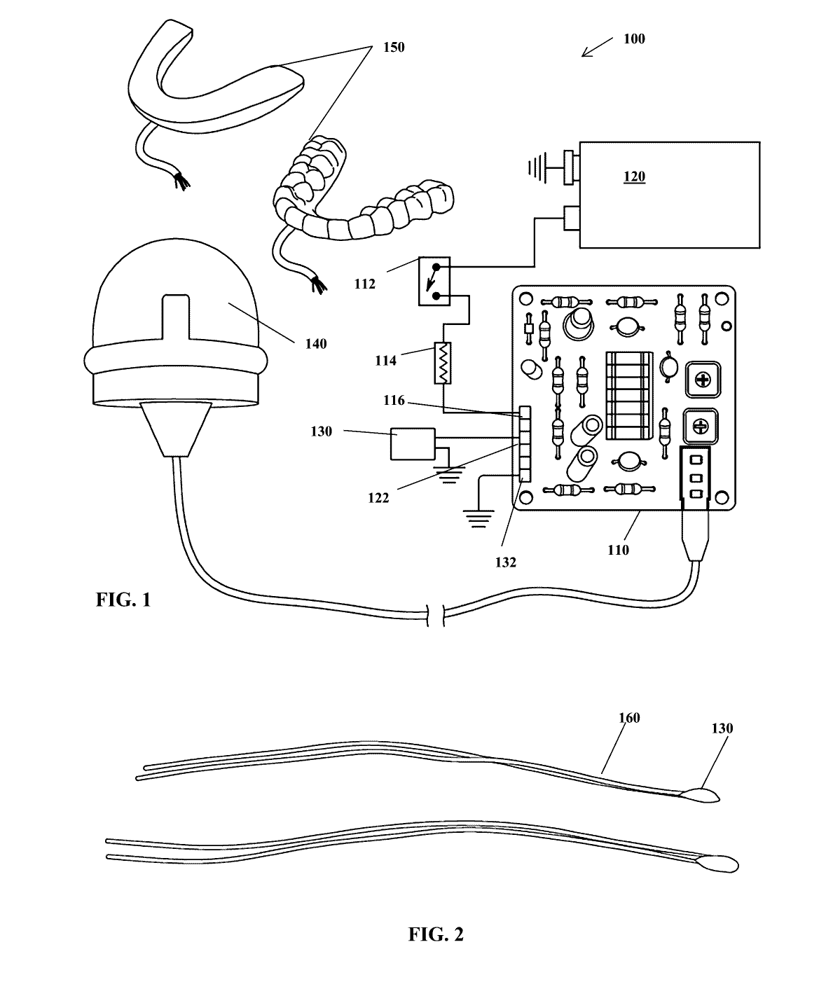

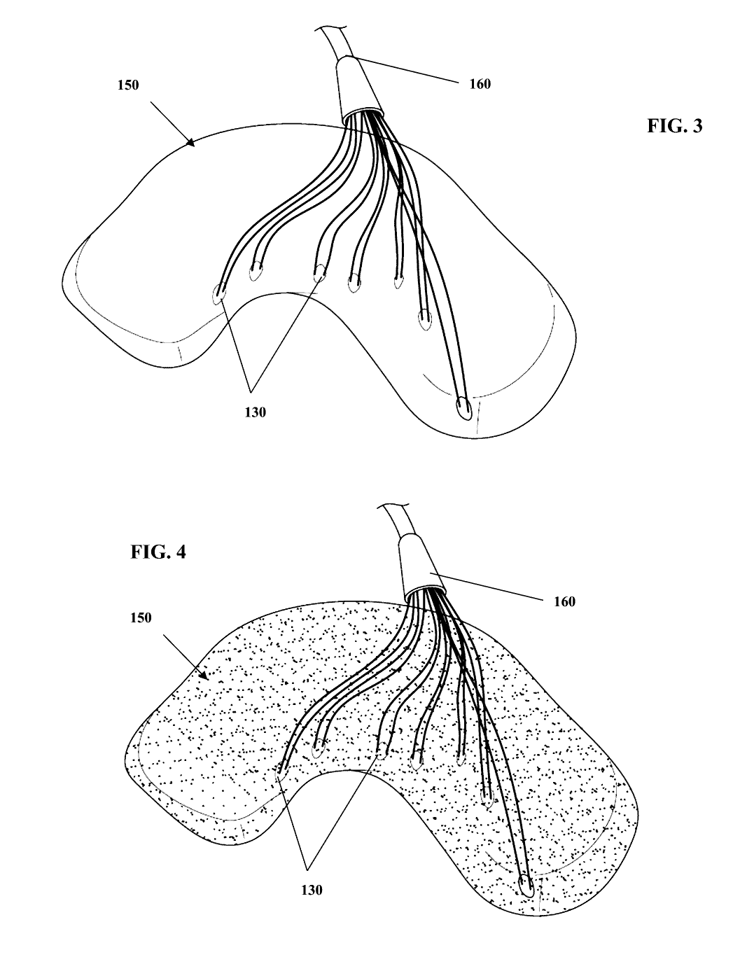

Generally speaking, the light wave treatment instrument 100 is shown in FIG. 1 and comprises a control unit 110 operably coupled to a power source 120, a light source 130, and a heart rate monitor 140. The light source 130 may be operably coupled to a light array 150. In one embodiment, the light array 150 may comprise an oral tray to optically dispose of the optical radiation, as shown in FIGS. 3 & 4. The light source 130 delivers optical radiation by way of an optical coupler 160 to an area of desired treatment that is synchronized to the heart beat or heart rate as determined by the heart rate monitor 140. The pulses or flashes from the light source 130 are triggered by the heart rate monitor 140 operably coupled to the control unit 110. The synchronization prevents the destruction of healthy cells and tissue during the optical radiation by the light source and reduce inflammation associated with the optical radiation. The synchronization stimulates cell renewal and rejuvenation by increasing oxygenation to the damaged tissue when the red blood cells are direct contact to the tissue by the cardiac cycle. “Synchronization” as used herein means cause things to happen at the same time and speed within an error of at least about 0.001 seconds.

In one embodiment, the optical coupler 160, as shown FIG. 2, may include the light source 130 at the end of the coupler 160. The light source 130 may be a Light Emitting Diode (LED), as further explained below.

The control unit 110 may comprise a switch 112, a resistor 114, and a power input 116. The switch 112 is operably coupled to the power source 120 and operates to turn the control unit 110 on and off. The control unit 110 further comprises an output 122 to the light source 130 and an electrical ground 132 for the heart rate monitor 140. The heart rate monitor 140 measures the cardiac cycle. The cardiac cycle refers to a complete heartbeat from its generation to the beginning of the next beat, and so includes the diastole, the systole, and the intervening pause. The frequency of the cardiac cycle is described by the heart rate, which is typically expressed as beats per minute. Each beat of the heart involves five major stages. The first two stages, often considered together as the “ventricular filling” stage, involve the movement of blood from the atria into the ventricles. The next three stages involve the movement of blood from the ventricles to the pulmonary artery (in the case of the right ventricle) and the aorta (in the case of the left ventricle).

The first stage, “early diastole,” is when the semilunar valves (the pulmonary valve and the aortic valve) close, the atrioventricular (AV) valves (the mitral valve and the tricuspid valve) open, and the whole heart is relaxed. The second stage, “atrial systole,” is when the atrium contracts, and blood flows from atrium to the ventricle. The third stage, “isovolumic contraction” is when the ventricles begin to contract, the AV and semilunar valves close, and there is no change in volume. The fourth stage, “ventricular ejection,” is when the ventricles are contracting and emptying, and the semilunar valves are open. During the fifth stage, “isovolumic relaxation time”, pressure decreases, no blood enters the ventricles, the ventricles stop contracting and begin to relax, and the semilunar valves close due to the pressure of blood in the aorta.

Throughout the cardiac cycle, blood pressure increases and decreases. The cardiac cycle is coordinated by a series of electrical impulses that are produced by specialised pacemaker cells found within the sinoatrial node and the atrioventricular node. The cardiac muscle is composed of myocytes which initiate their own contraction without the help of external nerves (with the exception of modifying the heart rate due to metabolic demand). Under normal circumstances, each cycle takes at least about 0.8 seconds or about 1.25 Hz. Thus, the optical radiation emitting by the light source to the light array 150 will be at least every 0.8 seconds or about or about 1.25 Hz. Alternatively, as the heart rate increases or decreases, the cycle may be between 0.01 seconds and 1.90 seconds or the light pulse is between about 100 Hz and 0.52 Hz. Alternatively, the cycle may be between 0.10 and 0.90 seconds or the light pulse is between about 10 Hz and 1.11 Hz. Alternatively the cycle may be between 0.40 and 0.90 or the light pulse is between about 2.5 Hz and 1.11 Hz. As the heart rate increases or decrease, the pulses of the light source 130 are synchronized and increased and decreased accordingly.

Light Source

In some embodiments, the light source 130 is emitted by arrays of Light Emitting Diodes (LEDs). Other light sources 130 may be used, such as lasers or superluminescent diodes (SLED or SLD), incoherent sources (flashlamp), radio frequency, microwave, or an x-ray source. A SLED is an edge-emitting semiconductor light source based on superluminescence. It combines the high power and brightness of laser diodes with the low coherence of conventional light-emitting diodes could suitably be employed. The character of the light emitted by light source right and left sides 1 and 3 will depend upon the nature of the LEDs or other light emitters in light source 130. It is generally desirable that the emitted light include light in the wavelength range of 620 nm to 1000 nm. In some embodiments the emitted light includes light having a wavelength in at least one of the following wavelength ranges: about 620 to about 890 nm and about 620 to about 680 nm. Light having wavelengths corresponding to one or more of the following ranges may be particularly effective: 653 nm to 684 nm; 667 nm to 674 nm; 650 nm to 673 nm; 612 nm to 746 nm.

The most important factors influencing light penetration in tissue are wavelength, super pulsing, power, intensity, tissue contact and compression. Other factors to consider are polarization and coherence.

The light is substantially monochrome in some embodiments although this is not mandatory. Providing light sources that emit at multiple wavelengths allows for irradiation over multiple wavelengths for alternative treatments, diseases, and biological activity. The optical radiation may comprise incoherent radiation although this is not mandatory. The optical radiation may be delivered at pulsed rates synchronized to the heart beat and at suitable frequencies and duty cycles.

Invisible infrared light can be clinically effective. In some embodiments in which the emitted light includes infrared light, the emitted light also includes bright visible light. The bright visible light deters users from looking into the light source when it is operating, provides a perceptible indication that the apparatus is operating, and may be useful in properly positioning the device. The visible light may be, but is not necessarily in a wavelength range that is beneficial for light therapy. In some embodiments, the ratio of the intensities of the visible and infrared components of the optical radiation part or less visible light to 5 parts or more infrared light.

The treatment area and desired light characteristics will vary from patient to patient. A physician, dentist or other therapist can determine a light treatment regime for a patient and set up light wave treatment instrument 100 to operate light emitters in light source 130 to provide the desired treatment.

In alternative embodiments, the light source is operably coupled to a light pulse generator. The light pulse generator is operably coupled and synchronized to the heart rate monitor. The light pulse generator includes an electronic circuit operably coupled with the control unit. The light pulse generator includes a repetition rate (frequency), delay, width and amplitude control.

The light array may align the light sources to the specific array of treatment. For example, the light array may align LED's to a corresponding area of treatment for periodontal disease in a patient.

Mechanism of Optical Radiation therapy

Photobiomodulation and photobiostimulation using non-coherent light occurs through three biochemical mechanisms. These are (i) wound healing, tissue repair and prevention of tissue death; (ii) relief of inflammation in chronic diseases and injuries with its associated pain and edema; (iii) relief of neurogenic pain and some neurological problems.

For low power visible light to have any effect on a living biological system, the photons must be absorbed by electronic absorption bands belonging to some molecular chromophore or photoacceptor. One approach to finding the identity of this chromophore is to carry out action spectra. This is a graph representing biological photoresponse as a function of wavelength, wave number, frequency, or photon energy, and should resemble the absorption spectrum of the photoacceptor molecule. The fact that a structured action spectrum can be constructed supports the hypothesis of the existence of cellular photoacceptors and signaling pathways stimulated by light.

The second important consideration involves the optical properties of tissue. Both the absorption and scattering of light in tissue are wavelength dependent (both much higher in the blue region of the spectrum than the red), and the principal tissue chromophore (hemoglobin) has high absorption bands at wavelengths shorter than 600 nm. For these reasons, there is a so-called “optical window”. Water begins to absorb significantly at wavelengths greater than 1150 nm. For these reasons, there is a so-called “optical window” in tissue covering the red and NIR wavelengths, where the effective tissue penetration of light is maximized.

In one embodiment, the light source is set to a wavelength and power setting such as to be absorbed by collagen. Wound healing is increased by the production of collagen. The beneficial effect of LLLT on wound healing can be explained by considering several basic biological mechanisms including the induction of expression cytokines and growth factors known to be responsible for the many phases of wound healing. Firstly, optical radiation between 632.8 nm to 670 nm increases both protein and mRNA levels of IL-1alpha.gif and IL-8 in keratinocytes. These are cytokines responsible for the initial inflammatory phase of wound healing. Secondly, the optical radiation can upregulate cytokines responsible for fibroblast proliferation and migration, such as bFGF, HGF and SCF. Thirdly, the optical radiation can increase growth factors such as VEGF, responsible for the neovascularization necessary for wound healing. Fourthly, TGF-B is a growth factor responsible for inducing collagen synthesis from fibroblasts, and may be upregulated by optical radiation. Fifthly, optical radiation can induce fibroblasts to undergo transformation into myofibloblasts, a cell type that expresses smooth muscle alpha.gif-actin and desmin, and has the phenotype of contractile cells that hasten wound contraction.

Control Unit

FIG. 1 illustrates a programmable control unit 110 that may be used to control the operation of light wave treatment instrument 100. The control unit 110 may be a separate, remote unit or may be directly connected to or integrated with light source 130. The control unit 110 may comprise a microprocessor, data store, power supply, clock and associated electronic circuitry. Control parameters are stored in the data store. The control unit 110 operates light source 130 according to the parameters in the data store that is operably coupled to the heart rate monitor 140. The parameters may specify when the light source 130 pulses light according to the patient's heart rate or heartbeat. The parameters may also specify one or more of: treatment duration; light intensity during the treatment; the rate at which light emitters are pulsed; the duty cycle at which the light emitters are pulsed; etc.

If light wave treatment instrument has sets of light emitters having the same characteristics (sets of LED that emit the same wavelength and pulse) or different characteristics (e.g. sets of LED that emit light at different wavelengths or sets of light emitters that illuminate at different locations) then separate control parameters may be provided to ensure all the light emitters have the same characteristics and emit at the same optical pulse synchronized to the heart beat monitor. In some embodiments, different sets of parameters are specified for different segments (intervals) of a light treatment. For example, light therapy treatments may be defined for a set of intervals each lasting from a few seconds to a 10 minutes or a fraction of an hour. Different parameters may be specified for each of the intervals. The intervals are not necessarily equal in length. In one embodiment, the intervals may be set to at least 1 minute, or at least 5 minutes, or about 1-10 minutes, or between about 1-30 minutes.

In some embodiments, different sets of parameters may be specified for different areas of light source 130. In some cases, some areas of light source 130 may be turned off because the treatment plan for a patient does not require light to be delivered at locations corresponding to those parts of the light source 130, or where sensors indicate that the condition of the tissue is treated according to a threshold or feedback mechanism. The feedback mechanism may be coupled to at least one sensor, either an optical sensor, thermal sensor, electrical sensor, piezo-electric sensor, temperature sensor, pressure sensor, magnetic sensor, and the like, which sends a signal back to the control unit indicating that the tissue beneath the light source has been treated or heated above a particular temperature or pressure.

A physician, dentist, or therapist may program a patient's treatment regimen into the control unit 110. This may be done, for example, with the aid of suitable software running on a computer that is in data communication with the control unit 110 or by way of a suitable user interface built into the control unit 110.

The control unit 110 may have one or more pre-set programs built in. As an alternative to, or as an aid to programming controller the physician, dentist, or therapist may select a pre-set program that is appropriate for controlling light wave treatment instrument 100 to deliver light to a patient according to a specified condition. In one embodiment, the condition may be periodontal disease, pain, inflammation, chronic degeneration, bacterial infection, and the like.

A typical treatment regimen provides a dose of light. Each of the doses of light may be delivered over a period lasting between a few minutes and an hour or so, depending on the interval or condition being treated. For example. ½ hour doses of light can be effective and are not unduly inconvenient for patients. A single daily dose appears to be as effective as dividing the same dose into multiple sessions delivered at different times during the day. Examples of possible treatment regimens are:

Enhancement of healing of the gums and collagen by applying light in 5 treatments per week for 12 weeks. Each treatment lasts ½ hour and illuminates the tissues of a patient's jaw with light having wavelengths of 660 nm and 840 nm. Alternatively, accelerating healing of gums and collagen by applying light in daily treatments for 21 days. Each treatment lasts between 20 minutes and one hour and illuminates the tissues of a patient's gum with light having a wavelength of 670 nm at the gum's surface.

The control unit 110 may maintain a log of treatments that have been delivered. For example, control unit 110 may log the date and time that each treatment was initiated, the duration of the treatment, and whether or not the treatment was completed. This log can be subsequently reviewed by a dentist, physician, or the like to evaluate whether or not the patient has complied with the prescribed treatment regimen.

The control unit 110 has a button or other suitable user patient interface that allows a patient to initiate a treatment according to previously-set parameters in the data store. The patient interface is preferably very simple such that minimal instruction is required to explain to a patient how to use light wave treatment instrument 100. The control unit 110 may include an audible or visual indicator that generates a signal to remind a patient that it is time for a treatment (or that a scheduled treatment is overdue).

A patient can use light wave treatment instrument at home or in another location by operating the control unit 110 to initiate delivery of a treatment.

The control unit 110 may comprise circuitry that monitors pulses, heartbeat, temperature at one or more locations in light source 130. The circuitry may monitor a signal modulated by a heartbeat sensor operably coupled with the light source 130. In the alternative, the control unit 110 may monitor the current and voltage driving LEDs in light source 130. The current/voltage relationship is temperature-dependent. Thus, by monitoring the current/voltage relationship the control unit 110 can determine whether the LED is at an undesirably high temperature. The control unit 110 may shut off or reduce current to light source 130 (or part of light source 130) when it detects that pulsing of the light source is out of synchronization with the heart beat monitor or if the light source 130 is undesirably high (or is trending towards being undesirably high). If light source 130 is equipped with a cooling fan then a programmable controller may optionally control the speed of the cooling fan in response to the monitored temperature.

The control unit 110 may be configured to maintain a log of treatments delivered by light wave treatment instrument 100. The log may be reviewed by a physician, dentist or technician to verify that light wave treatment instrument 100 has been used as prescribed by a patient. The log may track the times and durations of light therapy treatments delivered by light wave treatment instrument 100, heart rate and beat, and may also track other features such as operating temperatures, operational status and the like. The control unit 110 triggers the pulses of the light source 130 within an accuracy of 0.001 to about 0.0001 seconds.

If any synchronization is lost from the heart monitor, the control unit 110 will store the last previously recorded heart rate and pulse the light source between 0.1 and 10 seconds. If the heart rate is not calculated within 10 seconds to update the pulsing rate of the light source, an alarm may sound as to notify the patient of the lost heart rate signal and to re-apply the heart monitor or recalibrate the heart monitor to a better position to record the heart rate.

Synchronization can be treated as an appearance of some relation between the state vectors u(t) of two processes due to their interaction. A general synchronization is thus defined as the presence of a relation between the states of interacting systems, u2(t)=F[u1(t)]. If interacting systems are identical the states can coincide u1(t)=u2(t) and the synchronization is complete. If the parameters of coupled systems slightly mismatch, the states are close |u1(t)−u2(t)|≈0, but remain different. In classical sense of periodic, self-sustained oscillators, synchronization is usually defined as locking (entrainment) of the phases

φ_{n,m}=nΦ₁−mΦ₂=const, (1)

where n and m are integers, Φ₁, Φ2 are phases of the two oscillators and φ_{n,m} is the generalized phase difference.

Condition 1 is valid for quasi-periodic oscillators only. For more general forms of nonlinear oscillators (e.g. relaxation oscillators), a weaker condition for phase locking:

|nΦ₁−mΦ₂−δ|<const, (2)

In such cases, the m:n phase locking manifests as a variation of φ_{n,m} around a horizontal plateau. The amplitudes of phase synchronized oscillations can be quite different and need not be related.

For periodic oscillators, the condition of phase locking (1) is equivalent to the notion of frequency locking:

mf₁=mf₂, (3)

where f=(Φ)/2π and brackets mean time averaging. If n periods of the first oscillator have exactly the same duration as m periods of the second oscillator, the rhythms are m:n entrained. Synchronization of periodic oscillators thus means the appearance of phase locking and adjustment of frequencies. If we consider synchronization in the presence of noise, synchronization of chaotic systems, or synchronization of oscillators with modulated natural frequencies, phase and frequency locking may not be equivalent any more. One can distinguish between several forms of synchronization: frequency and phase locking, phase locking without frequency locking and frequency locking without phase locking.

For weak noise φ_{n,m} fluctuates in a random way around a constant value; the frequencies are then nearly locked, i.e., the condition of frequency locking 3 is fulfilled on average, n(f₁)=m(f₂). Strong noise can also cause phase slips. In such cases, the question synchronous or not synchronous cannot be answered in a unique way, but only treated in a statistical sense. Phase synchronization can be understood as an appearance of a peak in the distribution of the cyclic relative phase:

Ψ_{n,m}=φ_{n,m} mod 2π, (4)

and interpreted as the existence of a preferred stable value of phase difference between the two oscillators.

In case of synchronization with the heart beat coupling, the noise originates from measurements and external disturbances, but also from the fact that there are other subsystems that take part in the cardiovascular control and their influence is considered as noise in synchronization analysis with the pulsed optical radiation or light source. The heart beat monitor conducts synchronization with the light source pulses.

Heart Rate Monitor

Heart rate, or heart pulse, is the speed of the heartbeat measured by the number of poundings of the heart per unit of time—typically beats per minute (bpm). The normal resting adult human heart rate ranges from 60-100 bpm. A heart rate monitor is the easiest and most precise way to continuously measure your heart rate. Heart rate monitors, or HRM's,

A more precise method of determining heart rate involves the use of an electrocardiograph, or ECG (also abbreviated EKG). An ECG generates a pattern based on electrical activity of the heart, which closely follows heart function. Continuous ECG monitoring is routinely done in many clinical settings, especially in critical care medicine. On the ECG, instantaneous heart rate is calculated using the R wave-to-R wave (RR) interval and multiplying/dividing in order to derive heart rate in heartbeats/min. Multiple methods exist:

Alternative methods of measurement include pulse oximetry and seismocardiography. Pulse oximeters are being widely used for non-invasive, simultaneous assessment of haemoglobin oxygen saturation. They are reliable, accurate, relatively inexpensive and portable. Pulse oximeters are often used for estimating heart rate at rest and during exercise. The majority of these devices use an infrared source and a transistor photodetector for measuring the pulse. Seismocardiogram (SCG) is the recording of body vibrations induced by the heartbeat. SCG contains information on cardiac mechanics, in particular heart sounds and cardiac output.

A photoplethysmogram (“PPG”) is an optically obtained plethysmogram, a volumetric measurement of an organ. A PPG may be obtained by using a device known as a “pulse oximeter,” which illuminates a person's skin with a light emitting diode (“LED”) and measures light absorption or reflection with a photodiode. The photodiode produces a PPG signal indicative of the measured light absorption or reflection. Changes in this PPG signal may be used to detect the pulse rate of the heart. Pulse oximeters typically measure the rate of blood pulsations corresponding to the heartbeat of the patient by detecting the amount of red and infrared signals absorbed by oxygen in the blood.

An electrocardiogram (“ECG”) is a test used to monitor the electrical activity of the heart. An ECG signal is produced by an electric current flowing between electrodes contacting different sites on a person's body. An ECG signal can be processed along with a PPG signal to determine a person's blood pressure.

During the past two decades a number of relatively inexpensive portable heart monitors have been developed, which operate through use of PPG signals and/or ECG signals. Such heart monitors enable a person to view and/or record his/her heart pulse rate and/or blood pressure during active exercise and without visiting a medical facility. Patents directed to heart monitors include the following U.S. Pat. No. 5,316,008 of Suga, et al., issued May 31, 1994; U.S. Pat. No. 5,865,755 of Golub, issued Feb. 2, 1999; U.S. Pat. No. 6,599,251 of Chen, et al., issued Jul. 29, 2003; U.S. Pat. No. 6,723,054 of Baruch, et al., issued Apr. 20, 2004; and U.S. Pat. No. 7,993,275 of Banet, et al., issued Aug. 9, 2011, which are all hereby incorporated by reference for all that is disclosed therein.

### EXAMPLES

The following examples are put forth so as to provide those of ordinary skill in the art with a complete disclosure and description of how the compositions, compositions, articles, devices, systems, and/or methods claimed herein are made and evaluated, and are intended to be purely exemplary and are not intended to limit the scope of compositions, compositions, articles, devices, systems, and/or methods. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Periodontal Examination and Treatment

Periodontal Examination is a simple and rapid screening tool that is used to indicate the level of examination needed and to provide basic guidance on treatment needed. The most commonly used screening method for the measurement of depth of the gingival crevice and the clinical attachment level is periodontal probing. The clinician, by measuring probing depths, can make assumptions of the state of health of the periodontium. Periodontal Screening and Recording (PSR) has been adopted by the American Academy of Periodontology (AAP) and the American Dental Association (ADA) to initiate the promotion, prevention, and early treatment of periodontal diseases.

Various types of periodontal probes can be used to measure pocket depths. Various types of periodontal probes can be used to measure pocket depths. Most traditional probes are marked with 1-millimeter (mm) increments with the 4 and 6 mm marking absent. Many dental hygiene clinical boards require proficiencies with a probe reading of 1, 2, 3, 5, 7, 8, 9, and 10 mm. Some probes have a black band indicating the 3, 5, 9, and 10-mm markings. Other probes are marked at 3 mm. The current probe used in this testing was the University of Michigan O Probe with Williams markings (1 mm, 2 mm, 3 mm, 5 mm, 7 mm, 8 mm, 9 mm, and 10 mm).

The procedure is as follows:

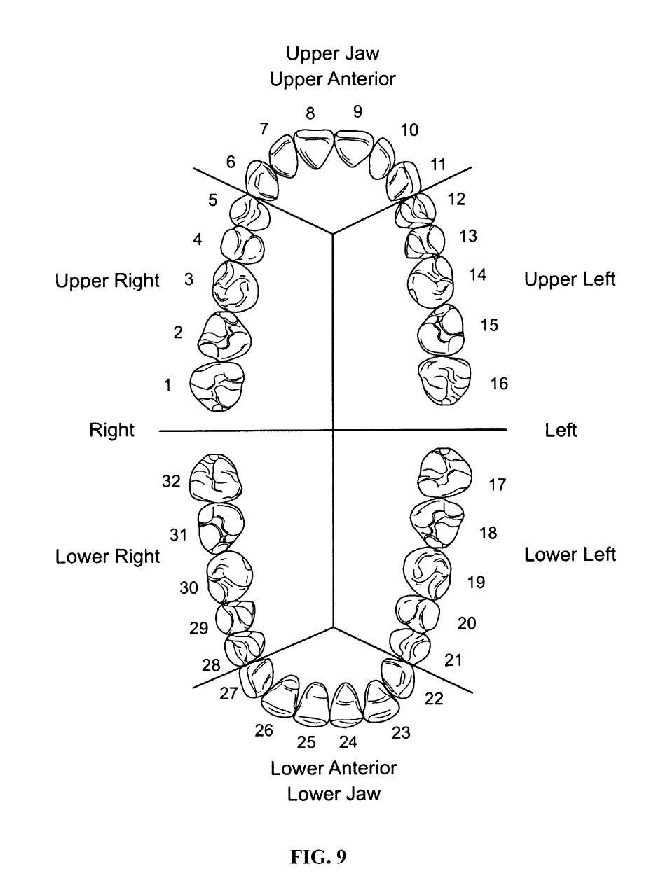

1. The dentition is divided into 6 sextants: upper right (1 to 5), upper anterior (6 to 11), upper left (12 to 16), lower left (17 to 21), lower anterior (27 to 22), lower right (28 to 32), as shown in FIG. 9.

2. All teeth in each sextant are examined (with the exception of 3rd molars).

3. For a sextant to qualify for recording, it must contain at least 2 teeth. (If only 1 tooth is present in a sextant, the score for that tooth is included in the recording for the adjoining sextant).

4. The Michigan O probe was used, which has a “ball end” 0.5 mm in diameter. Light probing force should be used (20-25 grams).

5. The probe is inserted into the sulcus between each tooth and the gingiva until resistance is felt, which indicates that the bottom of the periodontal pocket has been reached by the tip of the probe. The depth reading is then taken by noting the penetration of the probe, as visually gauged by the calibration mark which is judged to be closest to the height of the gingival margin. Standard practice involves taking six depth readings around each tooth at prescribed locations. Each reading is recorded, typically by verbally reporting it to an assistant who writes it by hand on a dental chart.

6. 0-3 mm in pocket depth is normal, any pocket depth greater than 3 mm may indicate periodontal disease, bone loss around the teeth or plaque induced inflammatory disease. Furcation involvement of the teeth is also recorded, as well as bleeding, Suppuration, and mobility of the teeth.

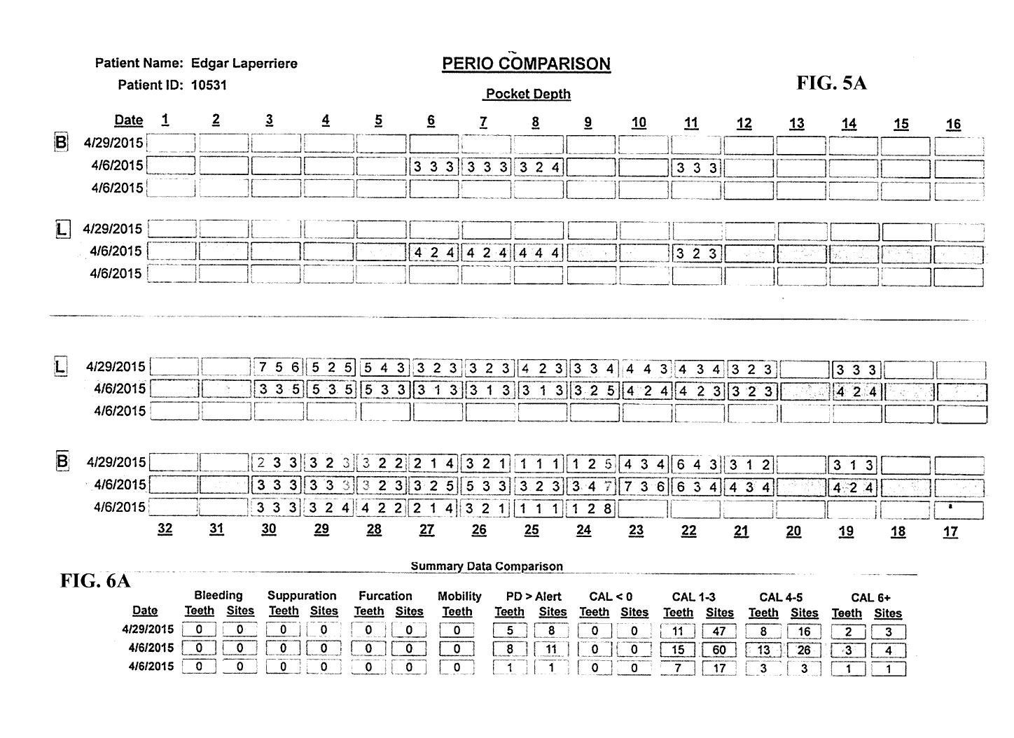

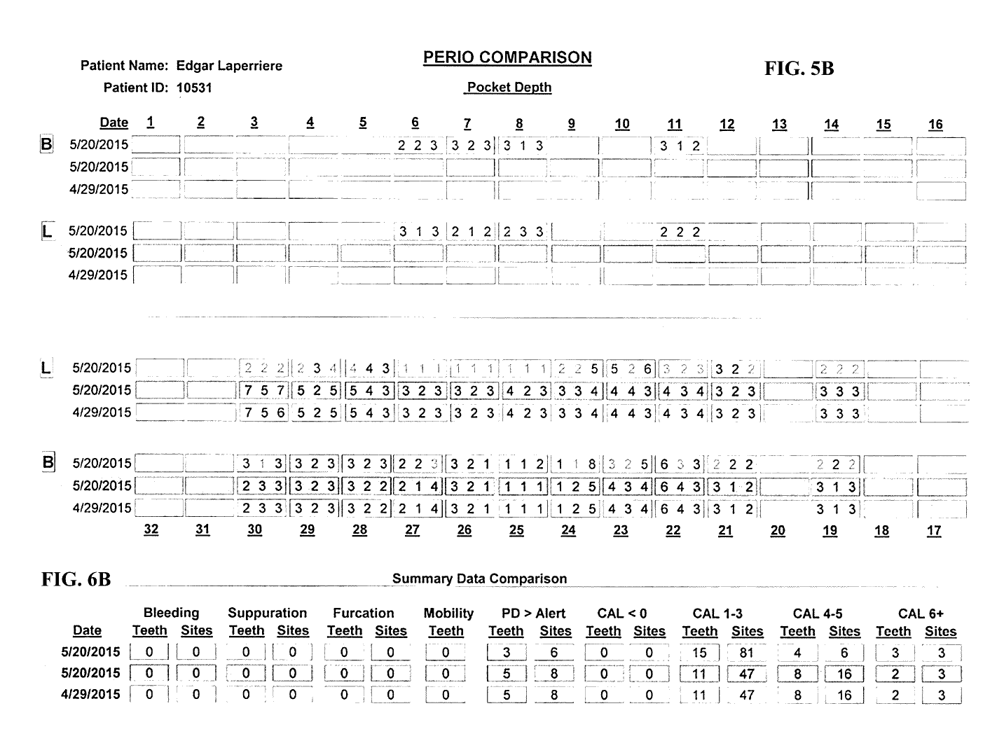

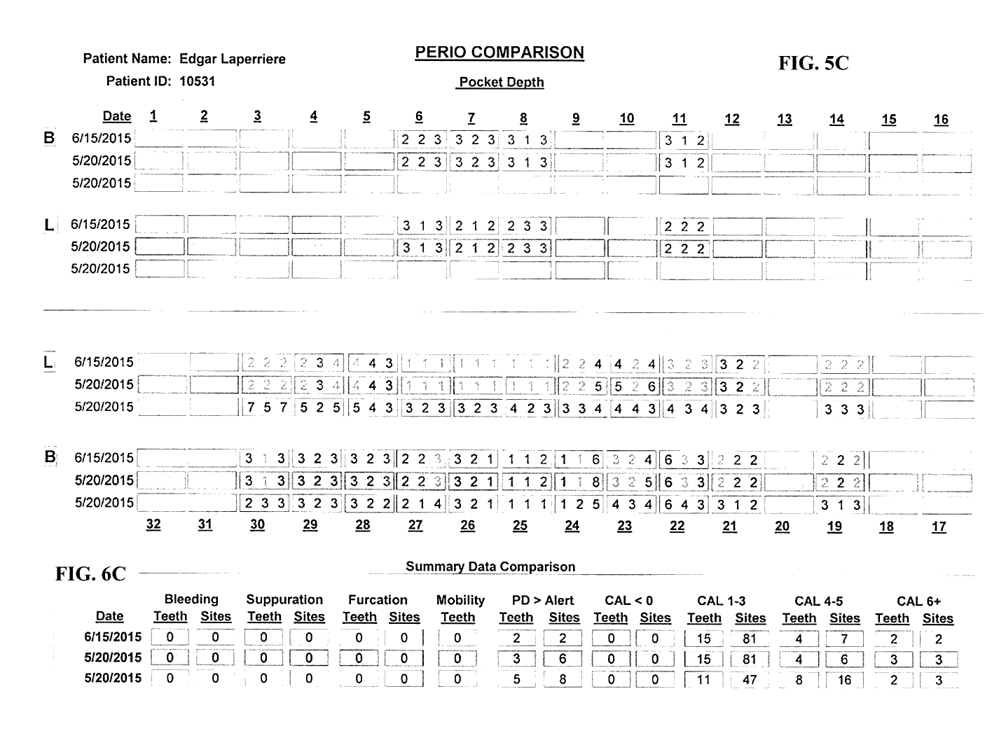

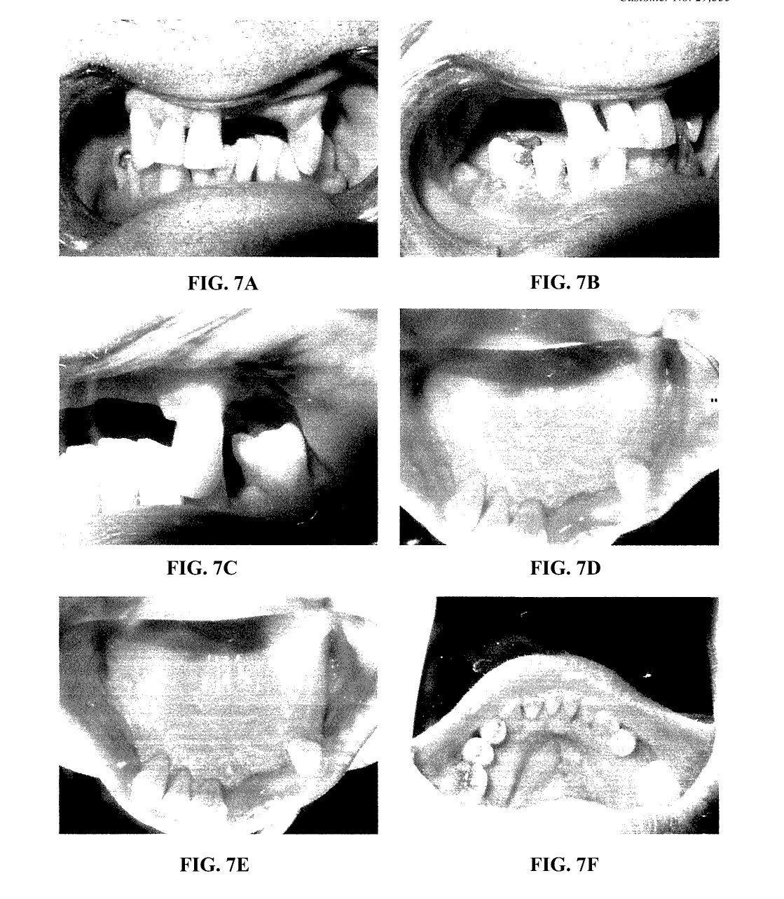

The results of the Periodontal Examination are shown in FIGS. 5A-6A and the associated photos in FIGS. 7A-7F. Images were taken associated with the Periodontal Examination. The examination for the examination on Apr. 6, 2015 and Apr. 29, 2015 showed periodontal disease and pocket depths showing periodontal disease.

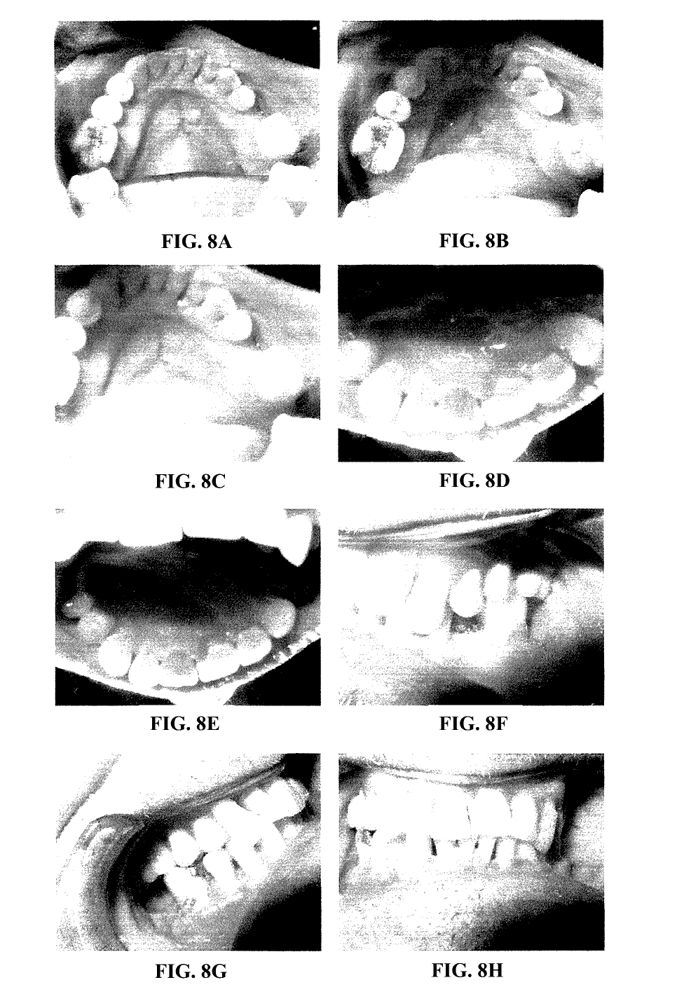

The light wave treatment instrument was employed with oral applications at 5 min intervals and a wavelength of 670 nm. The luminious intensity was a minimum 10.0 millicandelas (mcd) and typically around 21.0 mcd. The voltage was about 1.7 V and maximum at 2.4 V. The test current is about 20 mA and a view angle of about 140 degrees from the face of the LED. The optical radiation emitting by the light source to the light array 150 was at least every 0.8 seconds or about or about 1.25 Hz The positive results and reduction of the periodontal disease is shown in the results of the Periodontal Examination are shown in FIGS. 5B-6B, and the corresponding Summary Data comparison charts are shown in FIGS. 5C-6C, and the associated photos in FIGS. 8A-8H. The pocket depth decreased below 3 and improved the conditions of periodontal disease by the light wave treatment being synchronized to the heart beat monitor. As shown in FIG. 6C, the teeth with a pocket depth greater than 6 mm decreased to 2 teeth and the teeth with a pocket depth between 4-5 mm decreased to 4 teeth, when compared to FIG. 6A. This showed the decrease of periodontal disease and the effectiveness of the light wave instrument synchronization with the heart beat monitor in reducing inflammation, gum disease, and bone loss. No other treatments were implemented during this time period, such as antibiotics, gum scrapping or cleaning.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A light wave treatment instrument comprising:
a control unit operably coupled to a power source;
a light source operably coupled to the control unit operable to deliver pulses of optical radiation; and
a heart rate monitor operably coupled to the control unit, wherein the light source delivers pulses of optical radiation to an area of desired treatment synchronized to the heart beat determined by the heart rate monitor.

2. The light wave treatment instrument of claim 1, wherein the pulses of optical radiation from the light source are triggered by the heart rate monitor within an error of at least about 0.001 seconds.

3. The light wave treatment instrument of claim 2, wherein the light source is operably coupled to a coupler.

4. The light wave treatment instrument of claim 3, wherein the control unit comprises a switch.

5. The light wave treatment instrument of claim 4, wherein the pulses of optical radiation from the light source are between 0.01 and 0.10 seconds.

6. The light wave treatment instrument of claim 5, wherein the pulses of optical radiation are stopped by the control unit if the synchronization is off from the heart rate monitor.

7. The light wave treatment instrument of claim 6, wherein the light source is selected from the group consisting of: Light Emitting Diodes, lasers, superluminescent diodes, and incoherent sources.

8. The light wave treatment instrument of claim 7, wherein the light source includes a wavelength between 620 nm and 1000 nm.

9. A method of treating a patient comprising:
pulsing a light source synchronized with a heart rate monitor coupled to a patient; and
treating a condition of the patient.

10. The method of claim 9, wherein the condition is periodontal disease.

11. The method of claim 10, wherein the treating intervals are between 5 minutes.

12. The method of claim 11, wherein the light source includes a wavelength between 620 nm and 1000 nm.

13. The method of claim 12, wherein the pulsing step includes pulses of optical radiation between 0.01 and 0.10 seconds.

14. The method of claim 13, wherein the light source is selected from the group consisting of: Light Emitting Diodes, lasers, superluminescent diodes, and incoherent sources.

15. The method of claim 14, wherein the pulsing step is triggered by the heart rate monitor within an error of at least about 0.001 seconds.
